(19) United States
(12) Patent Application Publication
(10) Pub. No.: US 2026/0097394 A1
(43) Pub. Date: Apr. 9, 2026

(54) PREPARATION OF MFI ZEOLITES AND ZEOTYPES TO GENERATE STABLE PRODUCT SELECTIVITY FROM OLEFIN OLIGOMERIZATION

(71) Applicant: PURDUE RESEARCH FOUNDATION, West Lafayette, IN (US)

(72) Inventors: Rajamani P. Gounder, West Lafayette, IN (US); Evan Sowinski, West Lafayette, IN (US); Sanghyun Ahn, West Lafayette, IN (US)

(21) Appl. No.: 19/352,621

(22) Filed: Oct. 8, 2025

Related U.S. Application Data
(60) Provisional application No. 63/704,607, filed on Oct. 8, 2024.

(51) Int. Cl. B01J 29/40; B01J 35/77; B01J 37/03; B01J 37/04; B01J 37/30; C07C 2/12; C10G 50/00

(52) U.S. Cl. CPC B01J 29/40; B01J 35/77; B01J 37/031; B01J 37/036; B01J 37/04; B01J 37/30; C07C 2/12; C10G 50/00; B01J 2229/10; C07C 2529/40; C10G 2300/1092; C10G 2400/22

(57) ABSTRACT

MFI zeolite and methods for converting alkenes to higher liquid products. The method includes contacting one or more alkenes having about 2 to about 12 carbon atoms with a MFI zeolite having a silicon to aluminum ratio (Si:Al) of about 20 to about 100 and a crystallite size of about 0.001 μm to about 0.1 μm; and oligomerizing the one or more alkenes in the presence of the MFI zeolite to form an oligomer comprising one or more olefins having 4 to 26 carbon atoms. The MFI zeolite is synthesized with one or more organoammonium compounds.

### GOVERNMENT LICENSE RIGHTS

This invention was made with government support under Cooperative Agreement No. EEC-1647722 awarded by the National Science Foundation. The government has certain rights in the invention.

### BACKGROUND

### Field of the Invention

Embodiments provided herein relate to MFI zeolites and zeotypes. More particularly, embodiments provided herein relate to MFI (ZSM-5) zeolites and zeotypes for olefin oligomerization.

### Description of the Related Art

Light alkene oligomerization is a promising pathway to synthesize higher molecular weight hydrocarbons for transportation fuels and additives. Solid acids including supported phosphoric acid, acid resins, amorphous silica-alumina, and zeolites have been used in industrial oligomerization processes, with MFI zeolites garnering the most attention for these reactions due to its resistance to coke formation and high selectivity to linear olefins. MFI zeolites in their aluminosilicate composition are also known as ZSM-5. ZSM-5 is used in the Mobil Olefins to Gasoline and Distillate (MOGD) process, and there are several patents from the 1980s associated with this process.

Zeolites contain tetrahedral silicon atoms linked by oxygen, forming charge-neutral crystalline structures. The isomorphous substitution of trivalent aluminum atoms for tetravalent silicon introduces a negative charge imbalance that can be compensated by protons, which act as Brønsted acid sites for catalysis. Zeolites can also be synthesized with varying crystallite sizes and morphologies, with smaller crystallites leading to shorter diffusion pathlengths. Zeotypes are zeolite-like materials that include other elements in addition to silicon, aluminum, and oxygen.

Oligomerization is also accompanied by the undesired cracking (β-scission) reaction to form smaller hydrocarbons from oligomers. High selectivity is therefore achieved by preferentially slowing cracking reactions relative to oligomerization. Other synthetic variables that may affect the product distribution of olefin oligomerization in MFI include local aluminum proximity and the interplay between crystal size and bulk aluminum content. Another important consideration is how oligomerization rate and product selectivity change with time-on-stream. Generally, reaction rates decrease with time on-stream due to catalyst deactivation, which results from the build-up of heavier hydrocarbons within zeolite pores that introduce diffusion limitations and also from other mechanisms of deactivation such as active site poisoning or pore blocking. Additionally, product selectivities are observed to change with time-on-stream as zeolites deactivate, generally shifting the distribution towards primary products in the reaction network, such as hexene dimer products.

There is still a need, therefore, for new catalysts and catalyst synthesis that can lead to more stable time-on-stream operation, in terms of both the reaction rate and product selectivity.

### SUMMARY

A method for making MFI zeolites and the resulting MFI zeolites are provided. One particular method for making the MFI zeolite includes combining at least one organoammonium compound, a source of sodium, and water to form an aqueous solution; homogenizing the aqueous solution; adding a source of aluminum to the homogenized aqueous solution to form an intermediate agent; homogenizing the intermediate agent to form an aluminum-containing intermediate agent; adding a source of silicon to the aluminum-containing intermediate agent to form an aluminosilicate-containing intermediate agent; homogenizing the aluminosilicate-containing intermediate agent to form a synthesis gel; crystallizing the synthesis gel to form crystallized MFI zeolite solids; and then recovering the crystallized MFI zeolite solids.

The MFI zeolites (also referred to as ZSM-5 zeolites) have low aluminum contents (i.e., high Si/Al ratios) and small crystallite sizes. A preferred crystallite size is about 0.001 μm to about 0.1 μm, more preferably about 0.01 μm to about 0.05 μm. Other crystallite sizes (in microns) can range from a low of about 0.001, about 0.005, or about 0.01 to a high of about 0.05, about 0.075, or about 0.1. The MFI zeolites are particularly useful for oligomerization, and surprisingly applicable to ethene and propene oligomerization for making C4 to about C26 oligomers.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are, therefore, not to be considered limiting of its scope, for the invention may admit to other equally effective embodiments. It is emphasized that the figures are not necessarily to scale and certain features and certain views of the figures can be shown exaggerated in scale or in schematic for clarity and/or conciseness.

FIG. 1A depicts a SEM image of a commercial MFI (CBV2314).

FIG. 1B depicts a SEM image of the MFI sample SA1-001 that was synthesized according to one or more embodiments provided herein.

FIG. 1C depicts a SEM image of the MFI sample SA1-014 that was synthesized according to one or more embodiments provided herein.

FIG. 1D depicts a SEM image of the MFI sample SA2-002 that was synthesized according to one or more embodiments provided herein.

FIG. 2 depicts the time-on-stream conversion of propene oligomerization using the commercial CBV2314 and inventive MFI sample SA2-002 at different space velocities (reaction conditions were: 260° C., propene partial pressure 165 kPa, space velocities of 0.1 and 0.4 mol_{C3} mol_{H+}⁻¹ s⁻¹).

FIG. 3A shows the time-on-stream selectivity to primary oligomerization products (C₆, C₉, C₁₂, C₁₅) using MFI samples CBV2314 and SA2-002 (reaction conditions: 260° C., propene partial pressure 165 kPa, space velocities of 0.1 and 0.4 mol_{C3} mol_{H+}⁻¹ s⁻¹).

FIG. 3B shows the time-on-stream selectivity to oligomers with carbon number >7 (C₇₊) using MFI samples CBV2314 and SA2-002 (reaction condition: 260° C., propene partial pressure 165 kPa, space velocities of 0.1 and 0.4 mol_{C3} mol_{H+}⁻¹ s⁻¹).

FIG. 4A shows product distributions at iso-conversion at initial time-on-stream (1 h) and 21 h time-on-stream for CBV2314 (reaction condition: 260° C., propene partial pressure 165 kPa, space velocity 0.1 mol_{C3} mol_{H+}⁻¹ s⁻¹).

FIG. 4B shows product distributions at iso-conversion at initial time-on-stream (1 h) and 21 h time-on-stream for SA2-002 (reaction condition: 260° C., propene partial pressure 165 kPa, space velocity 0.4 mol_{C3} mol_{H+}⁻¹ s⁻¹).

FIG. 5 shows the time-on-stream conversion of propene oligomerization on SA2-002 at two temperatures (reaction condition: 260° C. and 280° C., propene partial pressure 165 kPa, space velocity of 0.4 mol_{C3} mol_{H+}⁻¹ s⁻¹).

FIG. 6A shows the time-on-stream selectivity to primary oligomerization products (C₆, C₉, C₁₂, C₁₅) for SA2-002 at 260° C. and 280° C.

FIG. 6B shows the time-on-stream selectivity to oligomers with carbon number >7 (C₇₊) for SA2-002 at 260° C. and 280° C.

FIG. 7 shows the time-on-stream conversion of propene oligomerization on SA2-002 at three different pressures (reaction condition: 260° C., propene partial pressure 45 kPa, 165 kPa and 602 kPa, space velocity of 0.4 mol_{C3} mol_{H+}⁻¹ s⁻¹).

FIG. 8A shows the time-on-stream selectivity to primary oligomerization products (C₆, C₉, C₁₂, C₁₅) for SA2-002 at 45 kPa, 165 kPa and 602 kPa.

FIG. 8B shows the time-on-stream selectivity to oligomers with carbon number >7 (C₇₊) for SA2-002 at 45 kPa, 165 kPa and 602 kPa.

### DETAILED DESCRIPTION

It is to be understood that the following disclosure describes several exemplary embodiments for implementing different features, structures, or functions of the invention. Exemplary embodiments of components, arrangements, and configurations are described below to simplify the present disclosure; however, these exemplary embodiments are provided merely as examples and are not intended to limit the scope of the invention. Additionally, the present disclosure can repeat reference numerals and/or letters in the various embodiments and across the figures provided herein. This repetition is for the purpose of simplicity and clarity and does not in itself dictate a relationship between the various embodiments and/or configurations. Moreover, the exemplary embodiments presented below can be combined in any combination of ways, i.e., any element from one exemplary embodiment can be used in any other exemplary embodiment, without departing from the scope of the disclosure.

Additionally, certain terms are used throughout the following description and claims to refer to particular components. As one skilled in the art will appreciate, various entities can refer to the same component by different names, and as such, the naming convention for the elements described herein is not intended to limit the scope of the invention, unless otherwise specifically defined herein. Further, the naming convention used herein is not intended to distinguish between components that differ in name but not function.

Furthermore, in the following discussion and in the claims, the terms “including” and “comprising” are used in an open-ended fashion, and thus should be interpreted to mean “including, but not limited to.” The phrase “consisting essentially of” means that the described/claimed composition does not include any other components that will materially alter its properties by any more than 5% of that property, and in any case does not include any other component to a level greater than 3 mass %.

Unless otherwise indicated, all numerical values are “about” or “approximately” the indicated value, meaning the values take into account experimental error, machine tolerances and other variations that would be expected by a person having ordinary skill in the art. It should also be understood that the precise numerical values used in the specification and claims constitute specific embodiments. Efforts have been made to ensure the accuracy of the data in the examples. However, it should be understood that any measured data inherently contains a certain level of error due to the limitation of the technique and/or equipment used for making the measurement.

The term “or” is intended to encompass both exclusive and inclusive cases, i.e., “A or B” is intended to be synonymous with “at least one of A and B,” unless otherwise expressly specified herein.

The indefinite articles “a” and “an” refer to both singular forms (i.e., “one”) and plural referents (i.e., one or more) unless the context clearly dictates otherwise. For example, embodiments using “an olefin” include embodiments where one, two, or more olefins are used, unless specified to the contrary or the context clearly indicates that only one olefin is used.

The term “oligomerization” refers to the formation of an oligomer (i.e. oligomer product) from molecules of lower relative molecular mass. The terms “oligomer” and “oligomer product” are used interchangeably herein and both refer to dimers, trimers, tetramers, and other molecular complexes having less than 26 repeating units. The oligomers provided herein are typically gases or liquids at ambient temperature, and can include low melting solids, including waxes, at ambient temperature. In some embodiments, the oligomers provided herein can have an atomic weight or molecular weight of less than 10,000 AMU (Da), such as about 5,000 or less, 1,000 or less, 500 or less, 400 or less, 300 or less, or 200 or less. The molecular weight of the oligomer, for example, can range from a low of about 50, 250 or 350 to a high of about 500, 3,000, 7,000, or 9,000 AMU (Da).

The terms “alkane” and “paraffin” both refer to any saturated molecule containing hydrogen and carbon atoms only, in which all the carbon-carbon bonds are single bonds and are saturated with hydrogen. Such saturated molecules can be linear, branched, and/or cyclic.

The terms “alkene” and “olefin” both refer to any unsaturated molecule containing hydrogen and carbon atoms only, in which one or more pairs of carbon atoms are linked by a double bond. Such unsaturated molecules can be linear, branched, or cyclic, and can include one, two, three or more pairs of carbon atoms linked by double bounds (i.e. mono-olefins, di-olefins, tri-olefins, etc).

The term “wt %” means percentage by weight, “vol %” means percentage by volume, “mol %” means percentage by mole, “ppm” means parts per million, and “ppm wt” and “ppmw” are used interchangeably and mean parts per million on a weight basis. All concentrations herein, unless otherwise stated, are expressed on the basis of the total amount of the composition in question.

Each of the appended claims defines a separate invention, which for infringement purposes is recognized as including equivalents to the various elements or limitations specified in the claims. Depending on the context, all references to the “invention” may in some cases refer to certain specific embodiments only. In other cases, it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions, when the information in this disclosure is combined with publicly available information and technology.

A detailed description of the MFI zeolites and methods for making and using the same will now be provided. In one or more embodiment, the MFI zeolites provided herein can be synthesized by combining at least one organoammonium compound, a source of sodium, and water to form an aqueous solution. The aqueous solution can then be homogenized and a source of aluminum can be added to the homogenized aqueous solution to form an intermediate agent. The intermediate agent can then be homogenized to form an aluminum-containing intermediate agent, to which a source of silicon is added to form an aluminosilicate-containing intermediate agent. The aluminosilicate-containing intermediate agent can be homogenized to form a synthesis gel, and then crystallized to form MFI zeolite solids that are recovered as the MFI zeolite solids.

The MFI zeolites (also referred to as ZSM-5 zeolites) have low aluminum contents (i.e., high Si/Al ratios) and small crystallite sizes. A preferred crystallite size is about 0.001 μm to about 0.1 μm, more preferably about 0.01 μm to about 0.05 μm. Other crystallite sizes (in microns) can range from a low of about 0.001, about 0.005, or about 0.01 to a high of about 0.05, about 0.075, or about 0.1.

The synthesis methods provided herein can easily scale-up to meet commercialization targets for MFI zeolites with smaller crystallite sizes that show product selectivity that remains more stable with time-on-stream as catalyst deactivation occurs, compared to commercially available MFI zeolites. This is a significant finding that will allow synthesizing the MFI zeolite catalyst at commercial scales to allow long-term operation of an oligomerization catalyst to produce a product stream of stable composition.

It has been surprisingly and unexpectedly discovered that such small MFI zeolites with the desired oligomerization performance can be synthesized using one or more organoammonium compounds, without the need of a surfactant (i.e., molecules that contain a hydrophobic functional group). For example, traditional surfactants such as polyethylenimine (PEIM), cetryltrimethylammonium (CTAB), dimethyloctadecyl[3-(trimethoxysilyl) propyl|ammonium chloride (TPHAC), and gemini-type quaternary ammonium surfactants (e.g., CₙH₂ₙ₊₁—N⁺(CH₃)₂-C₆H₁₂—N⁺(CH₃)₂-CₙH₂ₙ₊₁, where n=4-8) are not needed to synthesize the small MFI zeolites provided herein. In certain embodiments, minute amounts of one or more surfactants can be used, such as less than 1 wt %, 0.8 wt %, 0.6 wt %, 0.5 wt %, 0.4 wt %, 0.3 wt %, 0.2 wt %, 0.1 wt %, or less than 0.05 wt %, based on the total weight of the zeolite. Preferably, the small MFI zeolites are synthesized using zero surfactants.

It also has been surprisingly and unexpectedly discovered that time-on-stream product selectivity during propene oligomerization was found to be significantly more stable, as the crystallite size of MFI zeolites decreased. It also has been surprisingly and unexpectedly discovered that as MFI zeolite catalysts deactivate with time-on-stream, there is a shift in product selectivity toward primary oligomerization products (e.g., dimer and trimer products), and this becomes more pronounced as the crystallite size of MFI zeolites increase.

The MFI (ZSM-5) zeolites with low aluminum contents and small crystallite sizes can be synthesized through conventional techniques using one or more structure-directing agent (SDA) and/or organic structure-directing agents (OSDA). In at least one embodiment, the one or more structure-directing agent (SDA) and/or organic structure-directing agents (OSDA) can be or can include one or more organoammonium compounds. Suitable organoammonium compounds will not have a hydrophobic functional group. Suitable organoammonium compounds can also have 8 or less carbon centers along the length of the molecule. In certain embodiments, suitable organoammonium compounds can have less than 7, less than 6, less than 4, or less than 3 carbon centers along the length of the molecule. For example, suitable organoammonium compounds include, but are not limited to, tetrapropylammonium hydroxide (TPAOH) or N-butyl-N-methylpyrrolidinium hydroxide (BMPAOH), and combinations thereof.

In one embodiment, the MFI (ZSM-5) zeolites can be synthesized by adding one or more organoammonium compounds to water to form an aqueous solution; homogenizing the aqueous solution for a first time period; adding a source of aluminum to the homogenized aqueous solution to form an intermediate agent and homogenizing the intermediate agent for a second time period to form an aluminum-containing intermediate agent; adding a source of silicon to the aluminum-containing intermediate agent to form an aluminosilicate-containing intermediate agent and homogenizing the aluminosilicate-containing intermediate agent for a third time period to form a synthesis gel; subjecting the synthesis gel to a crystallization process to crystallize a MFI zeolite; and then recovering the solids (e.g., by centrifugation) followed by washing, drying and a high temperature air treatment and subsequent ion-exchange treatments to remove any unreacted reagents or SDA compounds, thereby recovering the acid-form zeolite.

The amount of the one or more organoammonium compounds per gram of silica can range from a low of about 0.5 gr, 0.6 gr, or 0.7 gr to a high of about 1.0 gr, 1.2 gr, or 1.4 gr. The amount of the one or more organoammonium compounds per gram of silica also can range from a low of about 0.3 gr, 0.4 gr, or 0.5 gr to a high of about 0.8 gr, 0.9 gr, or 1.0 gr.

The Na:Al ratio can range from a low value of about 0.1 to a high value of 0.3.

The Si:Al ratio can range from a low of about 25, 30, 40 to high of about 100, 200, 300. The Si:Al ratio also can range from a low of about 85, 125, or 165 to a high of about 275, 350, or 400. The Si:Al ratio also can be of from 100-400, 15-350, or 200-375.

The one or more organoammonium compounds can be or can include tetrapropylammonium hydroxide (TPAOH) or N-butyl-N-methylpyrrolidinium hydroxide (BMPAOH), or combinations thereof.

The source of aluminum can be aluminum hydroxide, aluminum sulfate, aluminum nitrate, aluminosilicate, and any combinations or derivatives thereof.

The source of silicon can be colloidal silica, a silicon alkoxide compound, fumed silica, amorphous silica, aluminosilicate, and any combinations or derivatives thereof.

The support material can have a surface area in the range of from about 10 m²/g to about 700 m²/g, a pore volume in the range of from about 0.1 cc/g to about 4.0 cc/g and an average particle size in the range of from about 0.01 μm to about 0.1 μm. More preferably, the support material can have a surface area in the range of from about 50 m²/g to about 500 m²/g, pore volume of from about 0.5 cc/g to about 3.5 cc/g and average particle size of from about 0.02 μm to about 0.05 μm. The surface area can range from a low of about 50 m²/g, 150 m²/g, or 300 m²/g to a high of about 500 m²/g, 700 m²/g, or 900 m²/g. The surface area also can range from a low of about 200 m²/g, 300 m²/g, or 400 m²/g to a high of about 600 m²/g, 800 m²/g, or 1,000 m²/g. The average pore size of the support material can range of from about 10 Å to 1000 Å, about 50 Å to about 500 Å, about 75 Å to about 350 Å, about 50 Å to about 300 Å, or about 75 Å to about 120 Å.

In at least one embodiment, the one or more organoammonium compounds can be or can include tetrapropylammonium hydroxide (TPAOH). The amount of TPAOH per gram of silica can range from a low of about a low of about 0.3 gr, 0.4 gr, or 0.5 gr to a high of about 0.8 gr, 0.9 gr, or 1.0 gr. The Si:Al ratio can range from a low of about 25, 30, 40 to high of about 100, 200, 300. The Si:Al ratio can also range from a low of about 85, 125, or 165 to a high of about 275, 350, or 400. The Si:Al ratio can also be 100-400, 15-350, or 200-375.

In at least one other specific embodiment, the one or more organoammonium compounds can be or can include N-butyl-N-methylpyrrolidinium hydroxide (BMPAOH) as an organic structure-directing agent (OSDA). The amount of BMPAOH per gram of silica can range from a low of about 0.5 gr, 0.6 gr, or 0.7 gr to a high of about 1.0 gr, 1.2 gr, or 1.4 gr. The Si:Al ratio can range from a low of about 25, 30, 40 to high of about 100, 200, 300. The Si:Al ratio also can range from a low of about 85, 125, or 165 to a high of about 275, 350, or 400. The Si:Al ratio also can be of from 100-400, 15-350, or 200-375.

Each of first, second and third time periods can be the same or can each be different. For example, each time period can range from about 1 second to about 48 hours; or about 1 to about 20 hrs; or about 2 to about 10 hrs; or about 3 to about 8 hrs; or about 3 to about 5 hrs; or about 3 to about 4 hrs.

The crystallization process preferably occurs at a temperature of about 130° C. to about 150° C., and also can range from a low of about 60° C., 70° C., 80° C. or 90° C. to a high of about 150° C., 180° C., 200° C. or 240° C. The crystallization process can also take place at about 100° C., 130° C., 140° C., 150° C., 160° C., 170° C., or 180° C.

The high temperature air treatment can occur at about 450 to about 550° C. The air treatment can also range from a low of about 400° C., 410° C., or 420° C. to a high of about 500° C., 550° C. or 600° C.

### Oligomerization

The zeolite catalyst, as described herein, can convert one or more light hydrocarbon alkenes to higher molecular weight oligomers. The light hydrocarbons or hydrocarbon feed stream can derive from natural gas, natural gas liquids, or mixtures of both. The hydrocarbon feed stream can be derived directly from shale gas or other formations. The hydrocarbon feed stream can also originate from a refinery, such as from a fluid catalytic cracking (FCC) unit, coker, steam cracker, and pyrolysis gasoline (pygas) as well as alkane dehydrogenation processes, for example, ethane, propane and butane dehydrogenation.

The hydrocarbon feed stream can be or can include one or more olefins having from 2 to 12 carbon atoms. The hydrocarbon feed stream can be or can include one or more linear alpha olefins, such as ethene, propene, butenes, pentenes and/or hexenes. The process is especially applicable to ethene and propene oligomerization for making C4 to about C26 oligomers.

The hydrocarbon feed stream can contain greater than about 65 wt % olefins, such as greater than about 70 wt. % olefins or greater than about 75 wt % olefins. For example, the hydrocarbon feed stream can contain one or more C2 to C12 olefins in amounts ranging from a low of about 50 wt %, 60 wt % or 65 wt % to a high of about 70 wt %, 85 wt % or 100 wt %, based on the total weight of the feed stream. The hydrocarbon feed stream also can include up to 80 mol % alkanes, for example, methane, ethane, propane, butane, and pentane; although the alkane generally comprises less than about 50 mol % of the hydrocarbon feed stream, and preferably less than about 20 mol % of the hydrocarbon stream.

The resulting oligomer(s) can be or can include one or more olefins having from 4 to 26 carbon atoms, such as 12 to 20 carbon atoms, or 16 to 20 carbon atoms. The resulting oligomers, for example, can include butene, hexene, octene, decene, dodecene, tetradecane, hexadecane, octadecene and eicosene and higher olefins, as well as any combinations thereof. The resulting oligomer(s) also can have less than about 5% aromatics and less than about 10 ppm sulfur. The resulting oligomer(s) also can have zero or substantially no aromatics and zero or substantially no sulfur.

The resulting oligomer(s) can be useful as precursors, feedstocks, monomers and/or comonomers for various commercial and industrial uses including polymers, plastics, rubbers, elastomers, as well as chemicals. For example, these resulting oligomer(s) are also useful for making polybutene-1, polyethylene, polypropylene, polyalpha olefins, block copolymers, detergents, alcohols, surfactants, oilfield chemicals, solvents, lubricants, plasticizers, alkyl amines, alkyl succinic anhydrides, waxes, and many other specialty chemicals.

The resulting oligomer(s) can be especially useful for production of diesel and jet fuels, or as a fuel additive. In certain embodiments, the resulting oligomer(s) can have a boiling point in the range of 170° C. to 360° C. and more particularly 200° C. to 300° C. The resulting oligomer(s) also can have a Cetane Index (CI) of 40 to 100 and more particularly 65 to 100. The resulting oligomer(s) also can have a pour point of −50° C. or −40° C.

The oligomerization process can be carried out using any conventional technique. The process can be carried out, for example, in a continuous stirred tank reactor, batch reactor or plug flow reactor. One or more reactors operated in series or parallel can be used. The process can be operated at partial conversion to control the molecular weight of the product and unconverted olefins can be recycled for higher yields.

Suitable reaction temperatures can be 200° C. or more, such as about 400° C. or more, about 450° C. or more, about 500° C. or more, about 525° C. or more, about 550° C. or more, and about 600° C. or more. The reaction temperature, for example, can range from about 200° C. to about 600° C., about 350° C. to about 575° C., or about 350° C. to about 550° C. Of course, lower reaction temperatures are also possible, which can range, for example, from a low of about 135° C., about 200° C. or about 225° C. to a high of about 350° C., about 400° C., or about 500° C.

Conventional oligomerization pressures can be used. For example, the reaction pressure can range from about 15 psig to about 4000 psig (1 Bar to 276 Bar), or about 15 psig to about 1500 psig (1 Bar to 103 Bar). The reaction pressure can also range from a low of about 15 psig (1 Bar), 500 psig (34.5 Bar) or 600 psig (41.4 Bar) to a high of about 1,000 psig (68.9 Bar), 1,200 psig (82.7 Bar), or 2,000 psig (138 Bar).

During oligomerization, the zeolite catalyst can be used alone or can be used with one or more promoters, and/or one or more co-catalysts or activators. The term promoter refers to any metal that can be added to the acid-form zeolite to provide another catalytically active compound, such as nickel. The terms “co-catalyst” and “activator” are used herein interchangeably and refer to any compound, other than the reacting olefin, that can added to the acid-form or metal/acid-form zeolite to further promote the reaction. For example, the following co-catalyst and/or activators can optionally be used: alumoxanes, aluminum alkyls, ionizing activators, which may be neutral or ionic, alumoxane compounds, modified alumoxane compounds, and ionizing anion precursor compounds that abstract one reactive, σ-bound, metal ligand making the metal complex cationic and providing a charge-balancing noncoordinating or weakly coordinating anion. Once the zeolite catalyst is deactivated with high molecular weight carbon, or coke, it can be regenerated using known techniques in the art, including for example, by combustion in air or nitrogen at a temperature of about 400° C. or higher.

### EXAMPLES

Embodiments discussed and described herein are further described with the following examples. Although the following examples are directed to specific embodiments, they are not to be viewed as limiting in any specific respect.

The SA1 series of MFI catalysts were crystallized in the presence of tetrapropylammonium hydroxide (TPAOH, 40%, Alfa Acsar) using synthesis mixtures prepared by combining tetraethylorthosilicate (TEOS, 98%, Sigma-Aldrich), aluminum isopropoxide (Al(O-i-Pr)₃, ≥98%, Sigma-Aldrich), 10 wt % sodium hydroxide solution (NaOH, 97 wt %, Sigma-Aldrich, dissolved in deionized water), and deionized water (18.2 MQΩ cm). The synthesis recipe was adapted from the synthesis reported by Song et al. [7]. The molar composition of the final synthesis mixture was αTPAOH·βAl₂O₃·γNa₂O·25SiO₂·δH₂O·100EtOH, where α, β, and γ are varied between 3≤α≤9, 0.25≤β≤0.5, 0.08≤γ≤0.16, and 200≤δ≤495, respectively. In typical synthesis, aluminum isopropoxide was mixed with solutions of TPAOH and NaOH in water in a perfluoroalkoxy alkane (PFA) jar. After being stirred at room temperature for 1 h, a given amount of TEOS was added and stirred 12 h at room temperature. The synthesis solution was then transferred to a 23 ml Teflon-lined stainless steel autoclave (Parr Instruments) and placed in a forced convection oven (Yamato DKN-402C) at 438 K under rotation (60 rpm) for up to 2 or 5 days. The synthesized solids were recovered via centrifugation, washed with deionized water until the pH of the supernatant reached a value below 9. The recovered solids were dried at 373 K for 24 h, and treated in flowing dry air (99.999% UHP, Indiana Oxygen) at 853 K for 10 h (0.0167 K s⁻¹). The detailed chemical compositions of the synthesis mixture used in each SA1 series MFI catalyst are listed in Table 1.

TABLE 1
Chemical compositions of the gels used in the
synthesis of SA1 series MFI catalysts studied.

| | Molar ratios | | | | | Crystallization |
|---|---|---|---|---|---|---|
| Sample | TPA | Al₂O₃ | Na₂O | SiO₂ | H₂O | time (days) |
| SA1-001 | 9 | 0.5 | 0.08 | 25 | 495 | 5 |
| SA1-002 | 9 | 0.25 | 0.08 | 25 | 495 | 5 |
| SA1-003 | 9 | 0.5 | 0.08 | 25 | 495 | 2 |
| SA1-004 | 5 | 0.25 | 0.08 | 25 | 495 | 2 |
| SA1-005 | 5 | 0.5 | 0.08 | 25 | 495 | 2 |
| SA1-006 | 5 | 0.5 | 0.08 | 25 | 300 | 2 |
| SA1-007 | 5 | 0.5 | 0.16 | 25 | 495 | 2 |
| SA1-008 | 5 | 0.5 | 0.16 | 25 | 300 | 2 |
| SA1-009^{b} | 3 | 0.5 | 0.16 | 25 | 495 | 2 |
| SA1-010^{b} | 3 | 0.5 | 0.16 | 25 | 300 | 2 |
| SA1-011^{b} | 3 | 0.5 | 0.08 | 25 | 495 | 2 |
| SA1-012^{b} | 3 | 0.5 | 0.08 | 25 | 300 | 2 |
| SA1-013 | 4 | 0.5 | 0.08 | 25 | 300 | 2 |
| SA1-014 | 4 | 0.5 | 0.16 | 25 | 300 | 2 |
| SA1-015^{c} | 4 | 0.5 | 0.08 | 25 | 200 | 2 |
| SA1-016^{c} | 4 | 0.5 | 0.16 | 25 | 200 | 2 |
| SA1-017 | 4 | 0.5 | 0.16 | 25 | 495 | 2 |
| SA1-018 | 4 | 0.5 | 0.08 | 25 | 495 | 2 |
| SA1-019^{b, c} | 3 | 0.5 | 0.16 | 25 | 300 | 2 |
| SA1-020^{b, c} | 3 | 0.5 | 0.08 | 25 | 300 | 2 |

^{a} All the syntheses were performed under rotation (60 rpm) at 438K.
^{b}The product obtained is a mixture of the MFI phase and amorphous phases.
^{c}Performed using pure MFI zeolite as a seed.

The SA2 series of MFI catalysts were crystallized using N-butyl-N-methylpyrrolidinium hydroxide (BMPAOH) as the organic structure-directing agent (OSDA). The synthesis of the OSDA and of the zeolite was adapted from the report of Gallego et al. [8]. Molar ratio of the synthesis solution was 0.4BMPAOH·αAl₂O₃·1.0SiO₂·15H₂O, where a is varied between 0.0025≤α≤0.033. In a typical synthesis of SA2-002, for example, 0.131 g of aluminum hydroxide (Al(OH)₃·1·0H₂O,≥98%, SPI pharma) and 6.2921 g of BMPAOH (41%, handmade) were dissolved in 3.444 g of deionized water (18.2 M (2 cm) in a PFA jar and the mixture was stirred for 1 h until clear solution under ambient conditions. Then, 6.00 g of colloidal silica (Ludox AS-40, 40 wt %, Sigma-Aldrich) was added to the mixture and stirred for 24 h under ambient conditions. The synthesis solution was then transferred to a 23 ml Teflon-lined stainless steel autoclave (Parr Instruments) and placed in a forced convection oven (Yamato DKN-402C) at 423 K and static condition for 14 days. The synthesized solids were recovered via centrifugation, washed with deionized water until the pH of the supernatant reached a value below 9. The recovered solids were dried at 373 K for 24 h, and treated in flowing dry air (99.999% UHP, Indiana Oxygen) at 853 K for 10 h (0.0167 K s⁻¹). The detailed chemical compositions of the synthesis mixture used in each SA2 series MFI catalyst are listed in Table 2.

TABLE 2
Chemical compositions of the gels used in the
synthesis of SA2 series MFI catalysts studied.

| | Molar ratios | | | | Crystallization |
|---|---|---|---|---|---|
| Sample | BMPA | Al₂O₃ | SiO₂ | H₂O | time (days) |
| SA2-001^{b} | 0.4 | 0.033 | 1.0 | 15 | 14 |
| SA2-002 | 0.4 | 0.017 | 1.0 | 15 | 14 |
| SA2-003 | 0.4 | 0.010 | 1.0 | 15 | 14 |
| SA2-004 | 0.4 | 0.005 | 1.0 | 15 | 14 |
| SA2-005^{c} | 0.4 | 0.017 | 1.0 | 15 | 14 |
| SA2-006^{c} | 0.4 | 0.010 | 1.0 | 15 | 14 |
| SA2-007^{c} | 0.4 | 0.005 | 1.0 | 15 | 14 |
| SA2-008 | 0.4 | 0.0033 | 1.0 | 15 | 14 |
| SA2-009 | 0.4 | 0.0025 | 1.0 | 15 | 14 |

^{a} All the syntheses were performed under static condition at 423 K.
^{b}The product obtained is a mixture of the MFI and Beta zeolite phases.
^{c}Carried out using tetraethylorthosilicate (TEOS, 98%, Sigma-Aldrich) instead of AS-40 as a Si source.

All MFI catalysts were converted into their NH₄-form by ion-exchange in aqueous 1M NH₄NO3 solution (98%, Sigma Aldrich) for 24 h under ambient conditions, followed by calcination in flowing dry air (1.67 cm³ s⁻¹ g_{cat}⁻¹, 99.999% UHP, Indiana Oxygen) at 773 K (0.0167 K s⁻¹) for 4 h to convert to their H-form.

The crystalline structure of synthesized materials was determined from powder X-ray diffraction (XRD) patterns measured on a Rigaku SmartLab X-ray diffractometer with a Cu Kα source (λ=0.154 nm) operated at 1.76 kW. Diffraction patterns were measured from 4-40° 2θ. Zeolite micropore volumes were calculated from Ar adsorption isotherms collected at 87 K for H-MFI samples in a Micromeritics ASAP 2020 Surface Area and Porosity Analyzer by finding the minimum of the semilogarithmic plot of σ(V_{ads})/σ(In(P/P₀)) versus In (P/P₀).

Elemental compositions of the samples were analyzed using inductively coupled plasma-optical emission spectroscopy (ICP-OES) with a Thermo Scientific iCAP 7000 Plus Series ICP-OES. Aqueous samples were prepared by dissolving ca. 0.02 g of solid in 2.5 g of hydrofluoric acid (48 wt %, Alfa Acsar). After >24 h, 1 g of HNO3 (70 wt %, Sigma-Aldrich) was added and diluted with 50 g of deionized water. The acidic site density of each sample was quantified by ammonia temperature programmed desorption (NH₃-TPD) of NH₄⁺-form samples on a Micromeritics AutoChem II 2920 Chemisorption analyzer and an Agilent 5973N mass selective detection (MSD) system. In TPD experiments, NH₄-form samples were held at 323 K for 0.5 h under flowing He (˜1×10⁻⁵ mol s⁻¹, 99.999%, Indiana Oxygen). The temperature was increased to 873 K (at 0.167 K s⁻¹) while quantifying the NH₃ desorbed.

Crystal sizes of the MFI zeolite samples were estimated with scanning electron microscopy (SEM) on Teneo (FEI) microscope operating at 2 kV. Prior to imaging, samples were coated with platinum to reduce charging of the insulating materials.

Propene reactions were performed in a stainless-steel reactor (9.5 mm i.d.) equipped with a concentric thermowell (K-type thermocouple) extending through the axial center of the reactor with the tip in the center of the catalyst bed to monitor temperature. In a typical experiment, 0.05-0.15 g of NH₄-form zeolite was pelletized and sieved to retain particles of fixed diameter (180-250 μm), and diluted with SiO₂ (Sigma-Aldrich, high purity grade 180-250 μm) in zeolite/SiO₂ weight-ratios of 0.03-0.33. This mixture was supported in the reactor by quartz-wool plugs and stainless-steel rods on both sides. The reactor was held within a furnace equipped with a temperature controller. Prior to oligomerization, an oxidative pretreatment (1.7×10⁻⁵ mol s⁻¹ flowing air (air zero, THC <1 ppm) and flowing 5.1×10⁻⁵ mol s⁻¹ helium (99.999%, Indiana Oxygen)) was performed by ramping the temperature to 823 K (0.05 K s⁻¹) to convert the sample into its H-form before cooling to reaction temperature (513-553 K). Reactant flows were generally composed of 30-70 mol % propene (99.99%, Matheson), 30 mol % methane as a diluent and internal standard (99.97%, Matheson), and balance helium as a diluent (99.999%, Indiana Oxygen). Space velocity was varied by changing the mass of catalyst loaded (0.05-0.15 g) and the flow rate of propene (7.5×10⁻⁶−2.0×10⁻⁵ mol s⁻¹).

As mentioned above, Table 1 lists the results from the syntheses using TPA⁺ ions as an organic SDA under the conditions described above. SA1-001 was first synthesized as nanocrystalline MFI zeolite. To optimize the zeolite synthesis recipes to lower the cost and increase the crystallization time, both to better meet scale-up commercialization targets, the molar ratio of each component in the synthesis mixture and crystallization time were varied. When the molar ratios of TPA, Na₂O, and water were fixed to 4, 0.16, and 300, respectively, MFI zeolite (SA1-014) was formed with solid yields above 25 wt % after crystallization time of 2 days.

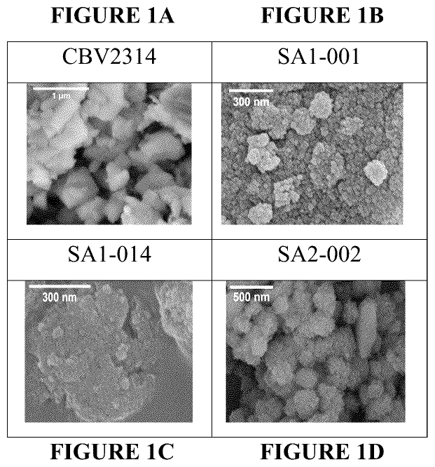

Measured Si/Al ratios and crystal sizes for the commercially available Al-MFI zeolite (CBV2314), SA1-001, SA1-14, and SA2-002 MFI zeolites are given in Table 3 below. All XRD patterns and micropore volumes collected were consistent with the MFI topology and typical of highly crystalline MFI structure (ca. 0.13 cm³ g⁻¹), respectively. SEM images of the samples are shown in FIG. 1.

TABLE 3
Catalyst properties of commercial and synthesized MFI

| | | Active site density, | |
|---|---|---|---|
| Sample | Si/Al | mol H⁺/g_{cat} | Crystal size, nm |
| CBV2314^{a} | 13 | 9.94 × 10⁻⁴ | 300 |
| SA1-001^{b} | 21 | 5.22 × 10⁻⁴ | 15 |
| SA1-014^{b} | 24 | 5.83 × 10⁻⁴ | 18 |
| SA2-002^{b} | 34 | 3.74 × 10⁻⁴ | 15 |

^{a}Obtained from Zeolyst International.
^{b}Synthesized at Purdue

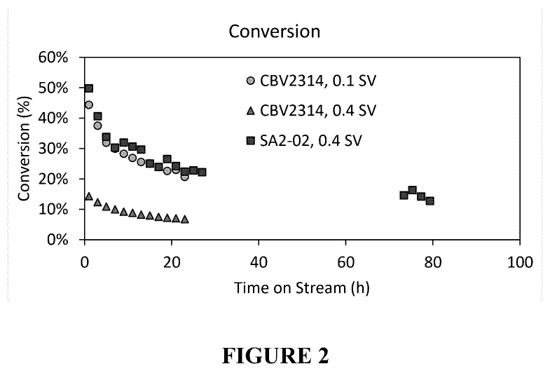

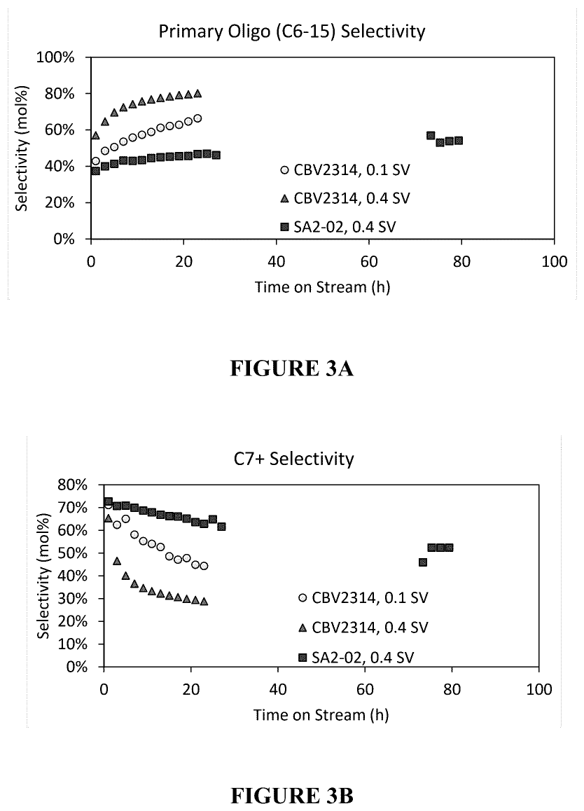

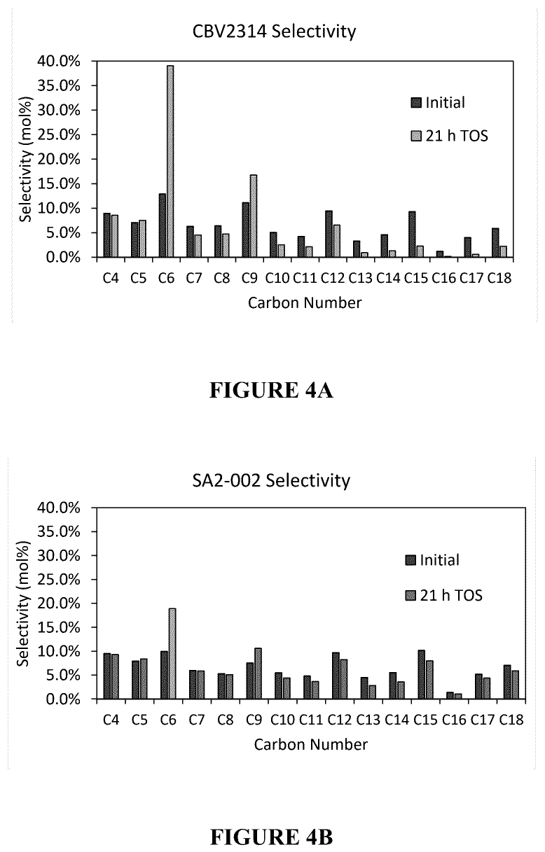

Conversion and selectivities of propene oligomerization are shown for the commercial Al-MFI zeolite (CBV2314) and SA2-002 in FIGS. 2-8. Cₙ (n=4, 5, . . . , 18) indicates the olefin products containing n carbon atoms. FIG. 2 shows that SA2-002 exhibits higher overall conversion at the same reaction conditions (260° C., space velocity (SV)=0.4 mol_{C3} mol_{H+}⁻¹ s⁻¹), relative to that of CBV2314. CBV2314 shows near iso-conversion with SA2-002 at a space velocity one-quarter (0.1 mol_{C3} mol_{H+}⁻¹ s⁻¹) that of SA2-002. Deactivation is observed in both catalysts in time-on-stream. However, SA2-002 shows stable product distribution as a function of reaction time relative to CBV2314 (FIG. 3). SA2-002 had lower selectivity to primary oligomerization products (C₆, C₉, C₁₂, C₁₅) compared to the commercial CBV2314 catalyst, and the selectivity did not increase as much with time-on-stream compared to CBV2314. SA2-002 shows higher selectivity to C₇₊ olefins compared to CBV2314. The initial C₇₊ selectivities are similar for the two catalysts, but SA2-002 shows slower decrease in selectivity towards C₇₊ with time-on-stream compared to that of CBV2314. Interestingly, it also appears that the C₇₊ selectivity deactivation tread is more linear with time-on-stream for SA2-002, rather than exponential decay observed in CBV2314. FIG. 4 shows total product distribution at initial time on stream (1 h) and at 21 h time-on-stream for CBV2314 and SA2-002 at iso-conversion. This confirms the observation that SA2-002 displays lower selectivity to primary oligomers (and thus greater selectivity to cracking products) compared to CBV2314, and greater selectivity to liquid products (C7+). It also shows that the change in product distribution from initial to later time on stream is more significant in CBV2314 than in SA2-002.

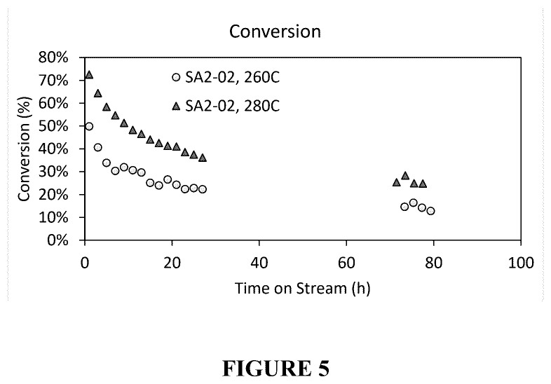

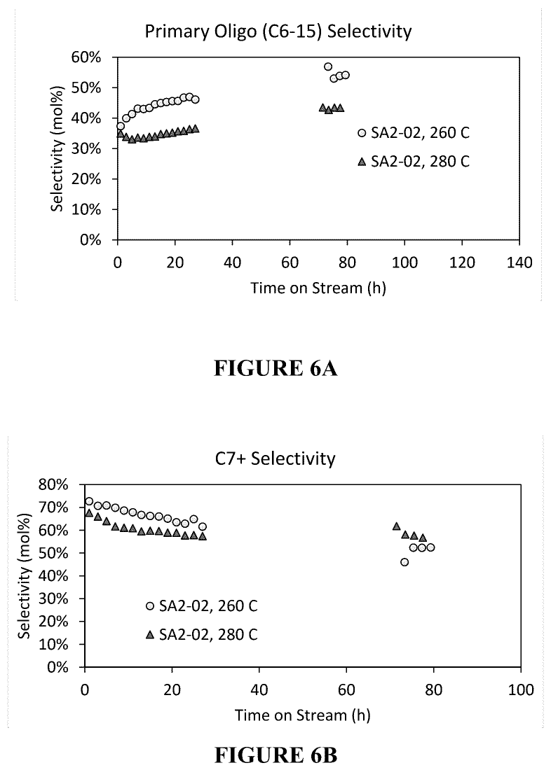

FIGS. 5 and 6 show conversion and selectivity (respectively) of SA2-002 at two different temperatures. As expected, the conversion increased with temperature. Selectivity to both primary oligomers and liquids (C₇₊) was similar, but slightly lower at higher temperature. This is likely due to increased rates causing cracking of primary oligomers and heavier products.

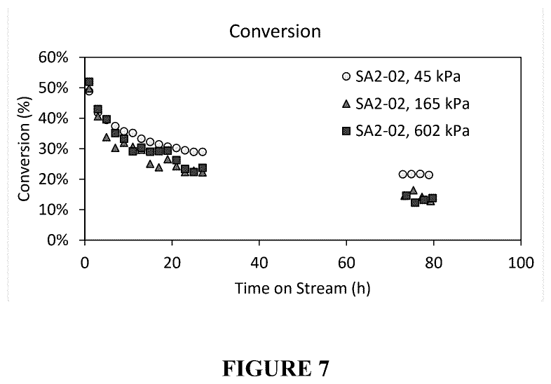

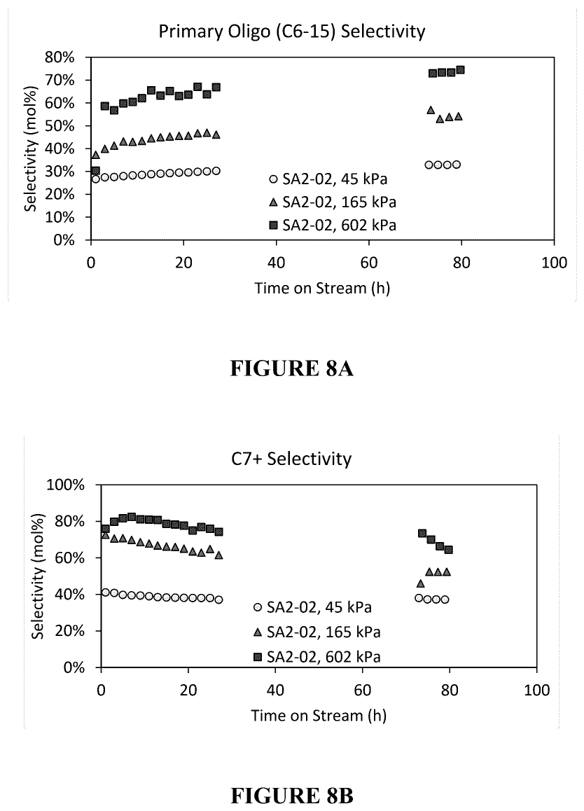

FIGS. 7 and 8 show conversion and selectivity (respectively) of SA2-002 at three different pressures. Conversion was similar in all cases, being slightly elevated at lower pressure. Selectivity to both primary oligomers and liquids differed significantly with pressure, with higher pressure yielding more primary oligomers and heavier products. In all cases, selectivity on SA2-002 was quite stable, with little change detected over the course of each experiment. In general, higher conversion led to greater stability in selectivity metrics. Additionally, process conditions such as temperature and pressure can be adjusted to tune product composition for a particular desired outcome.

The foregoing results show that the product selectivity from an olefin oligomerization catalyst can be stabilized to be at a near-constant composition over long times-on-stream, even during catalyst deactivation, via reduction in crystallite sizes of the zeolite catalysts. These catalysts can be manufactured quickly and economically, and offer significant new advantages for developing a continuous process for producing a liquid product from an oligomerization reactor that is intended for use in chemical and fuel markets.

Other specific embodiments include any of the Embodiments numbered 1 through 14 below:

Embodiment 1: A method for converting alkenes to higher liquid products, comprising: contacting one or more alkenes having 2 to 12 carbon atoms with a MFI zeolite having a silicon to aluminum ratio (Si:Al) of about 10 to about 100 and a crystallite size of about 0.001 μm to about 0.1 μm; oligomerizing the one or more alkenes in the presence of the MFI zeolite to form an oligomer comprising one or more olefins having 4 to 26 carbon atoms, wherein the MFI zeolite is obtained by: combining one or more organoammonium compounds, a source of sodium, and water to form an aqueous solution; homogenizing the aqueous solution; adding a source of aluminum to the homogenized aqueous solution to form an intermediate agent; homogenizing the intermediate agent to form an aluminum-containing intermediate agent; adding a source of silicon to the aluminum-containing intermediate agent to form an aluminosilicate-containing intermediate agent; homogenizing the aluminosilicate-containing intermediate agent to form a synthesis gel; and crystallizing the synthesis gel to form the MFI zeolite.

Embodiment 2: The method according to Embodiment 1, further comprising conducting an ion-exchange treatment to remove unreacted reagents, and then recovering the acid-form zeolite.

Embodiment 3: The method according to Embodiments 1 and/or 2, wherein the one or more organoammonium compounds are tetrapropylammonium hydroxide (TPAOH) or N-butyl-N-methylpyrrolidinium hydroxide (BMPAOH), or combinations thereof.

Embodiment 4: The method according to any Embodiment 1 to 3, wherein the one or more organoammonium compounds is tetrapropylammonium hydroxide (TPAOH).

Embodiment 5: The method according to any Embodiment 1 to 4, wherein the crystallizing the synthesis gel to form the MFI zeolite occurs at a temperature of about 90° C. to about 150° C.

Embodiment 6: The method according to any Embodiment 1 to 5, wherein a weight ratio of the one or more organoammonium compounds to silicon is 0.3 to 1.

Embodiment 7: The method according to any Embodiment 1 to 6, wherein the source of silicon is colloidal silica, a silicon alkoxide compound, fumed silica, amorphous silica, aluminosilicate, or any combinations thereof.

Embodiment 8: The method according to any Embodiment 1 to 7, wherein the source of aluminum is aluminum hydroxide, aluminum sulfate, aluminum nitrate, aluminosilicate, or derivatives thereof.

Embodiment 9: The method according to any Embodiment 1 to 8, wherein the one or more alkenes each have 2 to 6 carbon atoms.

Embodiment 10: The method according to any Embodiment 1 to 9, wherein the silicon to aluminum ratio (Si:Al) is about 20 to about 50.

Embodiment 11: The method according to any Embodiment 1 to 10, wherein the crystallite size is about 0.01 μm to about 0.05 μm.

Embodiment 12: The method according to any Embodiment 1 to 11, wherein the one or more alkenes are derived from natural gas, natural gas liquids, or mixtures of both.

Embodiment 13: The method according to any Embodiment 1 to 12, wherein the oligomer contains less than about 5% aromatics and less than about 10 ppm sulfur.

Embodiment 14: The method according to any Embodiment 1 to 13, wherein the oligomer has a boiling point in the range of about 170° C. to about 360° C.

In the above detailed description, the specific embodiments of this disclosure have been described in connection with its preferred embodiments. However, to the extent that the above description is specific to a particular embodiment or a particular use of this disclosure, this is intended to be illustrative only and merely provides a concise description of the exemplary embodiments. Accordingly, the disclosure is not limited to the specific embodiments described above, but rather, the disclosure includes all alternatives, modifications, and equivalents falling within the true scope of the appended claims. Various modifications and variations of this disclosure will be obvious to a worker skilled in the art and it is to be understood that such modifications and variations are to be included within the purview of this application and the spirit and scope of the claims.

All patents and patent applications, test procedures (such as ASTM methods, UL methods, and the like), and other documents cited herein are fully incorporated by reference to the extent such disclosure is not inconsistent with this disclosure and for all jurisdictions in which such incorporation is permitted.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, meaning the values take into account experimental error, machine tolerances and other variations that would be expected by a person having ordinary skill in the art.

The foregoing has also outlined features of several embodiments so that those skilled in the art can better understand the present disclosure. Those skilled in the art should appreciate that they can readily use the present disclosure as a basis for designing or modifying other methods or devices for carrying out the same purposes and/or achieving the same advantages of the embodiments disclosed herein. Those skilled in the art should also realize that such equivalent constructions do not depart from the spirit and scope of the present disclosure, and that they can make various changes, substitutions, and alterations herein without departing from the spirit and scope of the present disclosure, and the scope thereof is determined by the claims that follow.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A method for converting alkenes to higher liquid products, comprising:
contacting one or more alkenes having 2 to 12 carbon atoms with a MFI zeolite having a silicon to aluminum ratio (Si:Al) of about 10 to about 100 and a crystallite size of about 0.001 μm to about 0.1 μm;
oligomerizing the one or more alkenes in the presence of the MFI zeolite to form an oligomer comprising one or more olefins having 4 to 26 carbon atoms, wherein the MFI zeolite is obtained by:
combining one or more organoammonium compounds, a source of sodium, and water to form an aqueous solution;
homogenizing the aqueous solution;
adding a source of aluminum to the homogenized aqueous solution to form an intermediate agent;
homogenizing the intermediate agent to form an aluminum-containing intermediate agent;
adding a source of silicon to the aluminum-containing intermediate agent to form an aluminosilicate-containing intermediate agent;
homogenizing the aluminosilicate-containing intermediate agent to form a synthesis gel; and
crystallizing the synthesis gel to form the MFI zeolite.

2. The method of claim 1, further comprising conducting an ion-exchange treatment to remove unreacted reagents, and then recovering the acid-form zeolite.

3. The method of claim 1, wherein the one or more organoammonium compounds are tetrapropylammonium hydroxide (TPAOH) or N-butyl-N-methylpyrrolidinium hydroxide (BMPAOH), or combinations thereof.

4. The method of claim 1, wherein the one or more organoammonium compounds is tetrapropylammonium hydroxide (TPAOH).

5. The method of claim 1, wherein the crystallizing the synthesis gel to form the MFI zeolite occurs at a temperature of about 90° C. to about 150° C.

6. The method of claim 1, wherein a weight ratio of the one or more organoammonium compounds to silicon is 0.3 to 1.

7. The method of claim 1, wherein the source of silicon is colloidal silica, a silicon alkoxide compound, fumed silica, amorphous silica, aluminosilicate, or any combinations thereof.

8. The method of claim 1, wherein the source of aluminum is aluminum hydroxide, aluminum sulfate, aluminum nitrate, aluminosilicate, or derivatives thereof.

9. The method of claim 1, wherein the one or more alkenes each have 2 to 6 carbon atoms.

10. The method of claim 1, wherein the silicon to aluminum ratio (Si:Al) is about 20 to about 50.

11. The method of claim 1, wherein the crystallite size is about 0.01 μm to about 0.05 μm.

12. The method of claim 1, wherein the one or more alkenes are derived from natural gas, natural gas liquids, or mixtures of both.

13. The method of claim 1, wherein the oligomer contains less than about 5% aromatics and less than about 10 ppm sulfur.

14. The method of claim 1, wherein the oligomer has a boiling point in the range of about 170° C. to about 360° C.
